(19) United States
(12) Patent Application Publication
(10) Pub. No.: US 2018/0318152 A1
(43) Pub. Date: Nov. 8, 2018

(54) Tampon Disposal Packaging

(71) Applicant: Melane Garcia, Gilbert, AZ (US)

(72) Inventor: Melane Garcia, Gilbert, AZ (US)

(21) Appl. No.: 15/974,420

(22) Filed: May 8, 2018

Related U.S. Application Data
(60) Provisional application No. 62/503,145, filed on May 8, 2017.

(51) Int. Cl. A61F 13/551

(52) U.S. Cl. CPC A61F 13/5518; A61B 50/37; A61F 13/55185

(57) ABSTRACT

A tampon disposal packaging that can be used to secure both an un-used and used tampon has a wrapper body and an adhesive strip. The wrapper body has a primary wrapping layer, a secondary wrapping layer, and a sealable perimeter. In a first configuration, the primary wrapping layer is folded over to encase the secondary wrapping layer, the adhesive strip, and a tampon, wherein the sealable perimeter secures the edges of the primary wrapping layer together. The primary wrapping layer has a pocket portion that forms a pocket in conjunction with the secondary wrapping layer, allowing for the disposal of the tampon once the primary wrapping layer has been opened. Further, the primary wrapping layer has a flap portion to which the adhesive strip is connected. The flap portion can be folded over the secondary wrapping layer in order to close the pocket and secure the tampon within the pocket.

The current application claims a priority to the U.S. Provisional Patent application Ser. No. 62/503,145 filed on May 8, 2017.

### FIELD OF THE INVENTION

The present invention relates generally to feminine hygiene products. More specifically, the present invention is a tampon disposal packaging that is used to secure both an un-used and used tampon.

### BACKGROUND OF THE INVENTION

Tampons are a common feminine hygiene product that have been used by women for decades. While tampon packaging allows a tampon to be carried discretely, the packaging is often discarded after opening, as the packaging becomes torn and unusable. Thus, it is not clear how one should properly dispose of used tampons. Some manufacturers say that the tampons are safe to flush down the toilet. In reality, this is typically not the case, as flushing used tampons can lead to clogged toilets. Some say they should be disposed of in the trash, which is correct, but the only materials typically available in a bathroom that can be used to wrap up the used tampon is toilet paper or paper towels. Not only is the use of toilet paper or paper towels wasteful, but it is also not sanitary. Disposable bags have been designed for the disposal of tampons, but such bags are separate from the tampon and are often not readily available when one needs to discard a tampon. Additionally, such bags have to be purchased separately and the individual has to remember to take one with them, adding an additional item to carry and keep track of.

It is therefore an objective of the present invention to provide a tampon disposal packaging that can be used to secure both an un-used and used tampon. The tampon disposal packaging can be pre-packaged in a first configuration, wherein a wrapper body encases a tampon that has not been used. The wrapper body includes a first tab and a second tab that are used to open a primary wrapping layer of the wrapper body along a sealable perimeter in order to remove the tampon. The primary wrapping body opens to reveal a pocket that can be used to hold the used tampon and an adhesive strip that is used to seal the pocket closed, securing the tampon inside. As such, the user will not need to purchase a separate disposable bag to discard the tampon, as the user can simply hold on to the tampon disposal packaging until the tampon needs to be discarded. Ideally the tampon disposal packaging is manufactured from a biodegradable or compostable material in order to reduce the environmental impact of discarding the tampon disposal packaging. The tampon disposal packaging allows the user to still discretely carry an un-used tampon, while providing the additional disposal benefits.

### BRIEF DESCRIPTION OF THE DRAWINGS

FIG. 1 is a right side perspective view of the present invention.

FIG. 2 is a left side perspective view of the present invention, showing the opening into the pocket.

FIG. 3 is a front view of the present invention, showing the wrapper body in the first configuration, wherein a tampon is encased within the primary wrapping layer.

FIG. 4 is a rear view of the present invention, showing the wrapper body in the first configuration, wherein a tampon is encased within the primary wrapping layer.

FIG. 5 is a right side view of the present invention, showing the first half of the sealable perimeter being removably attached to the second half of the sealable perimeter, while the wrapper body is in the first configuration.

FIG. 6 is a perspective view of the present invention, wherein the first half of the sealable perimeter is being separated from the second half of the sealable perimeter in order to open the primary wrapping layer, wherein the tampon may be removed.

FIG. 7 is a front view of the present invention, wherein the wrapper body has been opened flat and the tampon has been removed.

FIG. 8 is an illustration showing a user placing the used tampon within the pocket formed by the primary wrapping layer and the secondary wrapping layer.

FIG. 9 is a front view of the present invention, showing the used tampon being placed within the pocket, and showing the protective layer of the adhesive strip being peeled back from the adhesive layer of the adhesive strip.

FIG. 10 is a front view of the present invention, showing the wrapper body in the second configuration, wherein the flap portion is folded over the secondary wrapping layer, and wherein the adhesive strip is adhered to the secondary wrapping layer to secure the opening and the pocket closed.

### DETAIL DESCRIPTIONS OF THE INVENTION

All illustrations of the drawings are for the purpose of describing selected versions of the present invention and are not intended to limit the scope of the present invention.

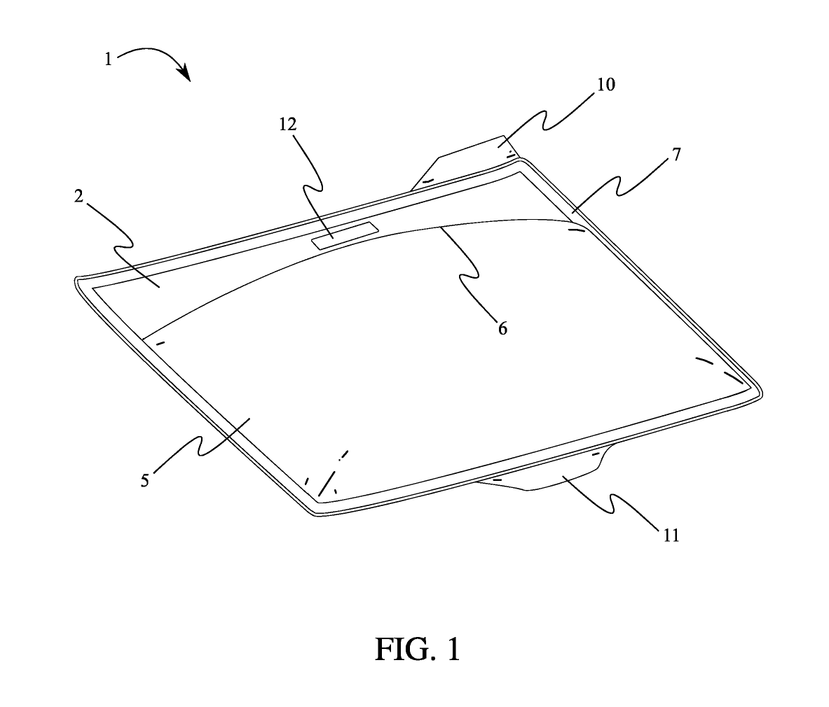

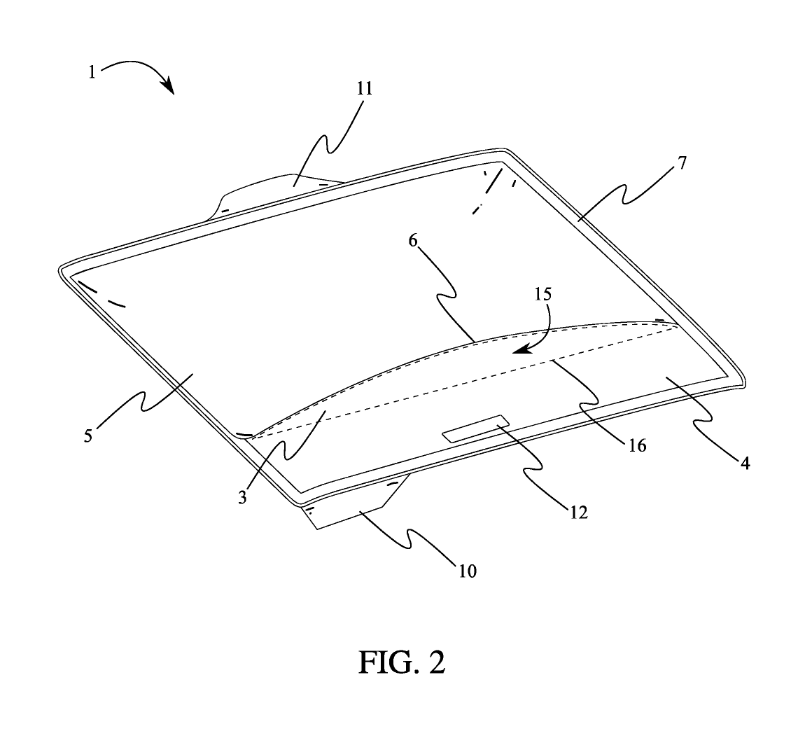

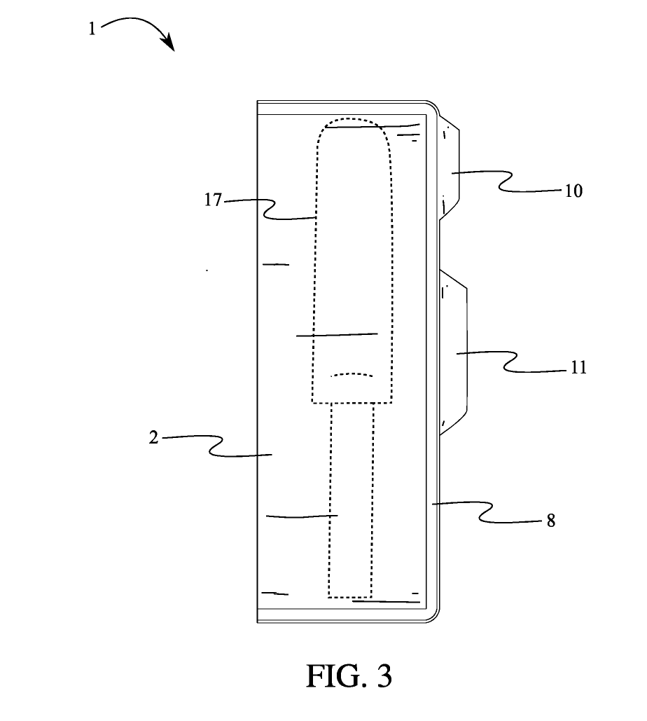

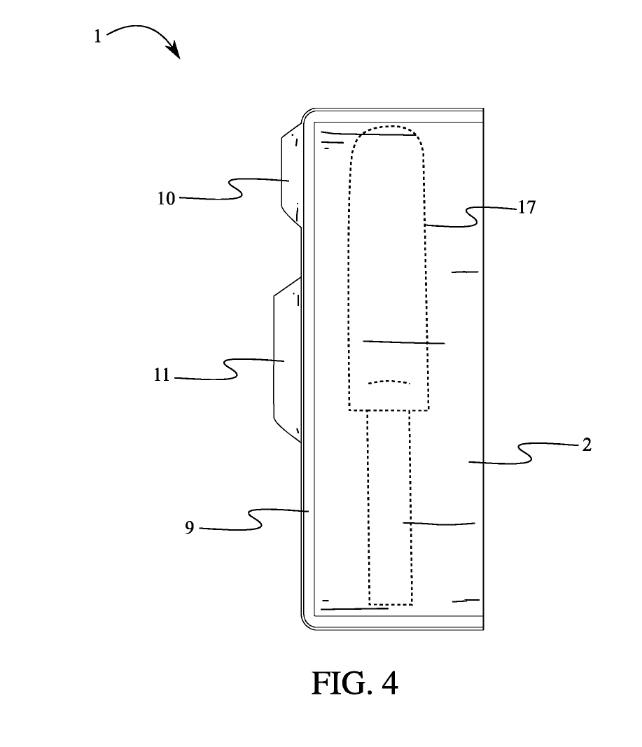

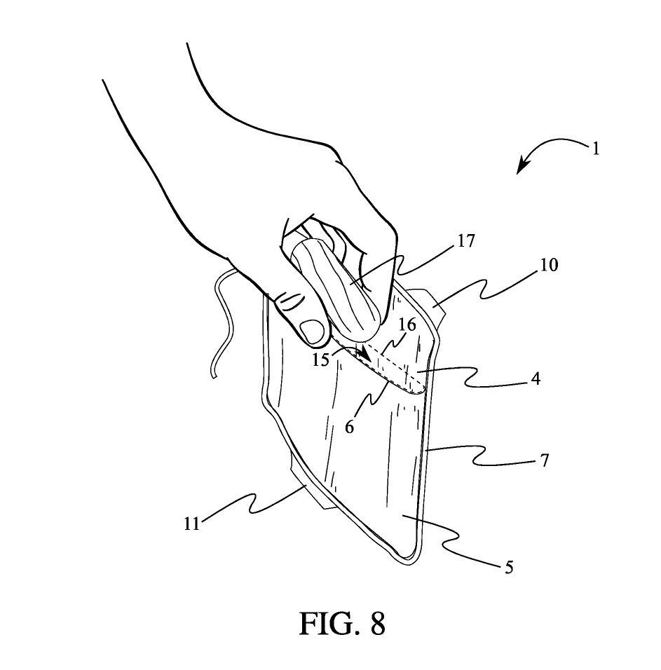

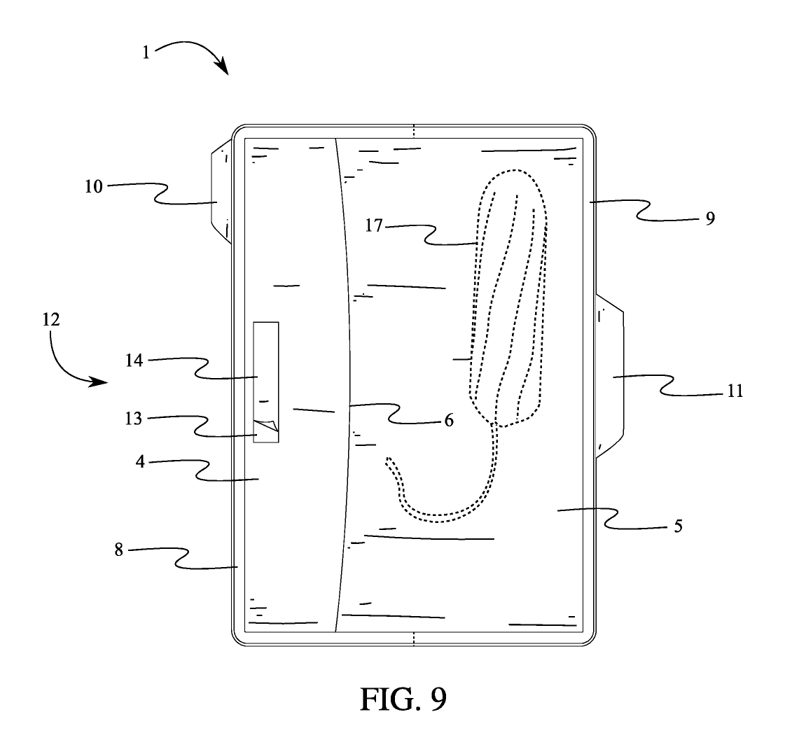

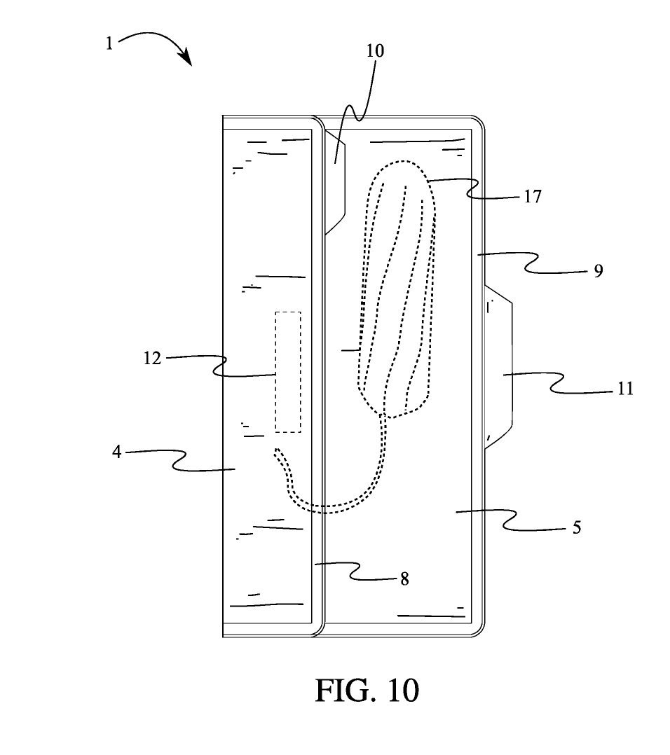

The present invention is a tampon disposal packaging that is used to secure both an un-used and used tampon. In reference to FIG. 1-2, the present invention comprises a wrapper body 1 and an adhesive strip 12. The wrapper body 1 can be folded in a first configuration, as depicted in FIG. 3-5, to secure a tampon 17 that has not yet been used, wherein the tampon 17 may be encased in the wrapper body 1 similar to a traditional tampon 17 packaging. Once the tampon 17 has been used and needs to be disposed of, the tampon 17 may be placed in a pocket 15 of the wrapper body 1, as depicted in FIG. 8-9. The wrapper body 1 may then be folded into a second configuration, as depicted in FIG. 10, wherein the adhesive strip 12 is used to seal the pocket 15 closed. Ideally the tampon disposal packaging is manufactured from a biodegradable or compostable material in order to reduce the environmental impact of discarding the tampon disposal packaging.

In reference to FIG. 1-2, the wrapper body 1 comprises a primary wrapping layer 2, a secondary wrapping layer 5, and a sealable perimeter 7. In the first configuration, the primary wrapping layer 2 is folded in half to enclose the tampon 17, wherein the sealable perimeter 7 secures the edges of the primary wrapping layer 2 together, as depicted in FIG. 3-5. The sealable perimeter 7 secures the edges of the primary wrapping layer 2 together in a temporary manner, such that the wrapper body 1 may be opened to release the tampon 17, while maintaining the integrity of the primary wrapping layer 2 and the secondary wrapping layer 5. The secondary wrapping layer 5 is secured to the primary wrapping layer 2 in order to form the pocket 15 in which the tampon 17 may be placed for disposal.

In reference to FIG. 1-2 and FIG. 7, the sealable perimeter 7 is perimetrically positioned about the primary wrapping layer 2, such that the edges of the primary wrapping layer 2 may be secured together when the primary wrapping layer 2 is folded in half. In some embodiments, the sealable perimeter 7 may be integrated with the primary wrapping layer 2, such that the primary wrapping layer 2 and the sealable perimeter 7 are constructed from a single piece of material. In other embodiments, the sealable perimeter 7 and the primary wrapping layer 2 may be separate pieces of material, wherein the sealable perimeter 7 is adjacently connected to the primary wrapping layer 2.

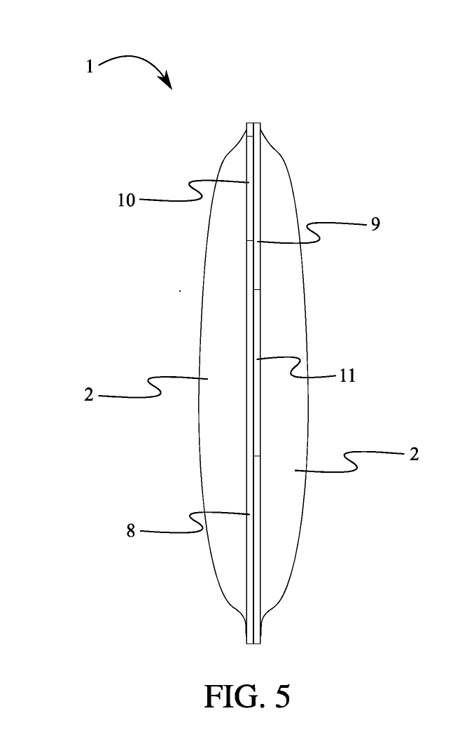

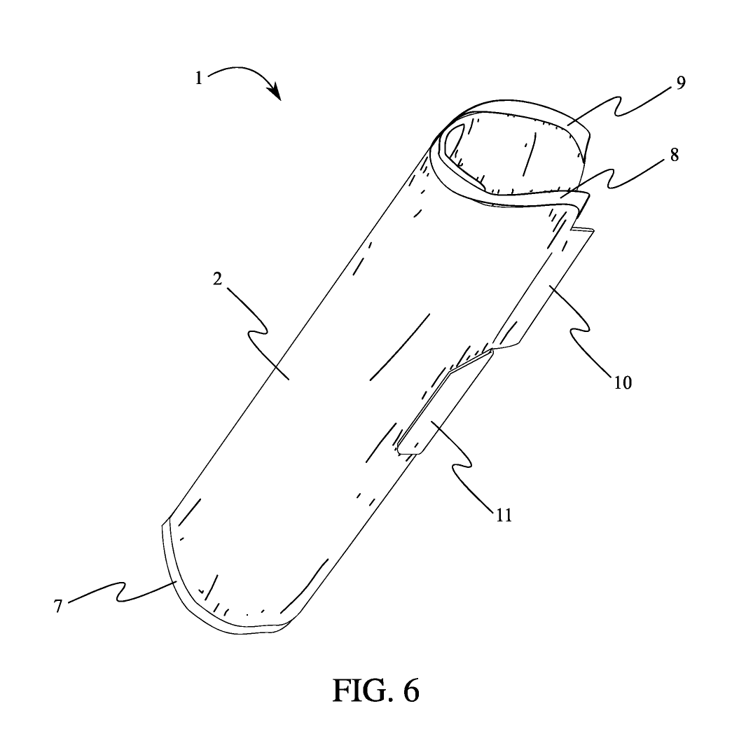

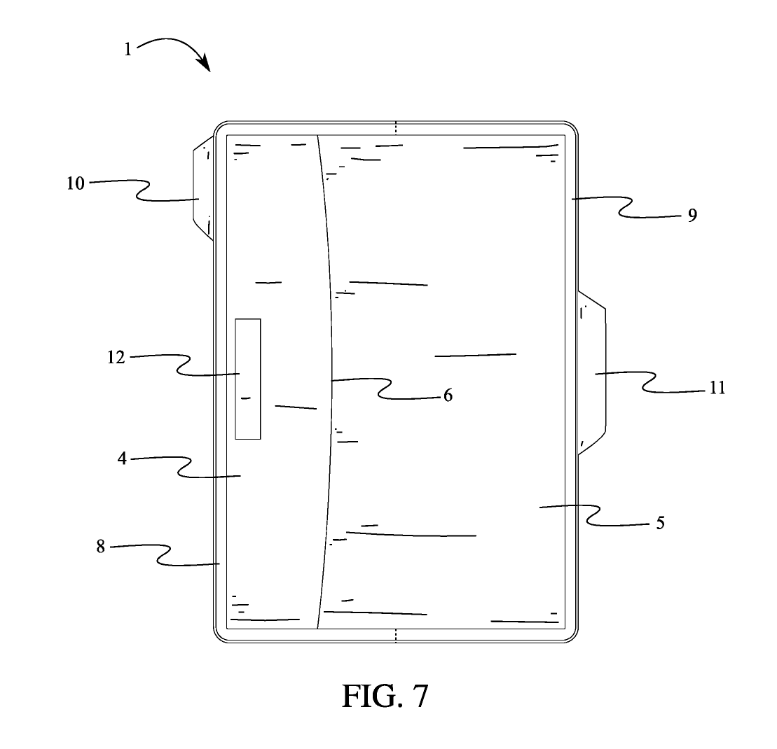

In further reference to FIG. 7, the sealable perimeter 7 comprises a first half 8 and a second half 9. In the first configuration, the first half 8 is folded over the second half 9 to enclose the secondary wrapping layer 5, the adhesive strip 12, and the tampon 17 within the primary wrapping layer 2. The first half 8 and the second half 9 are aligned with each other, wherein the first half 8 is removably attached to the second half 9. The detachable nature of the first half 8 and the second half 9 allows a user to open up the primary wrapping layer 2 to retrieve the tampon 17 without tearing, or otherwise damaging, the primary wrapping layer 2 or the secondary wrapping layer 5, as depicted through FIG. 5-7. Thus, the integrity of the pocket 15 is maintained for the future containment of the tampon 17.

The first half 8 may be removably attached to the second half 9 using a variety of methods to preserve the integrity of the primary wrapping layer 2 and the secondary wrapping layer 5. In some embodiments, the first half 8 may be adhered to the second half 9, such that the first half 8 is adhesively bonded to the second half 9 along an adhesive seamline. To unwrap the tampon 17 in such a configuration, the user may grasp the primary wrapping layer 2 on opposing sides of the adhesive seamline and apply sufficient force in opposite directions, such that the first half 8 and the second half 9 are pulled away from each other. As the user continues to apply force, the first half 8 and the second half 9 are progressively separated along the adhesive seamline until the primary wrapping layer 2 is opened flat.

In other embodiments, the first half 8 may be tearably attached to the second half 9, such that the first half 8 and the second half 9 form a perforated edge. To unwrap the tampon 17 in such a configuration, the user may grasp the primary wrapping layer 2 on opposing sides of the perforated edge and apply sufficient force in opposite directions, such that the first half 8 and the second half 9 are pulled away from each other. As the user continues to apply force, the first half 8 and the second half 9 are progressively separated along the perforated edge until the primary wrapping layer 2 is opened flat.

In yet other embodiments, the first half 8 and the second half 9 may be heat sealed, such that the first half 8 is thermally bonded to the second half 9 along a thermal seamline. To unwrap the tampon 17 in such a configuration, the user may grasp the primary wrapping layer 2 on opposing sides of the thermal seamline and apply sufficient force in opposite directions, such that the first half 8 and the second half 9 are pulled away from each other. As the user continues to apply force, the first half 8 and the second half 9 are progressively separated along the thermal seamline until the primary wrapping layer 2 is opened flat.

In reference to FIG. 2, the primary wrapping layer 2 comprises a pocket portion 3 and a flap portion 4; the pocket portion 3 forming one half of the pocket 15 and the flap portion 4 being used to secure the pocket 15 closed. The secondary wrapping layer 5 is positioned adjacent to the pocket portion 3 and is adjacently connected to the primary wrapping layer 2 about the sealable perimeter 7 to form the pocket 15. The flap portion 4 extends past the secondary wrapping layer 5, wherein the flap portion 4 can be folded over the secondary wrapping layer 5 in order to secure the pocket 15 closed.

In reference to FIG. 2 and FIG. 8, the secondary wrapping layer 5 comprises a non-affixed edge 6 which delineates an opening 16 to the pocket 15. As such, the non-affixed edge 6 is separated from the primary wrapping layer 2. The non-affixed edge 6 may be pulled away from the primary wrapping layer 2 in order to widen the opening 16, allowing for the tampon 17 to be more easily inserted into the pocket 15 when it is time to dispose the tampon 17. The non-affixed edge 6 is positioned adjacent to the flap portion 4, such that the flap portion 4 may be folded over the non-affixed edge 6 to seal both the opening 16 and the pocket 15 closed once the tampon 17 has been positioned within the pocket 15, as depicted in FIG. 10.

In the second configuration of the wrapper body 1, wherein the flap portion 4 is folded over the secondary wrapping layer 5, the adhesive strip 12 is used to secure the flap portion 4 to the secondary wrapping layer 5. As such, the adhesive strip 12 is adjacently connected to the flap portion 4, wherein the adhesive strip 12 and the secondary wrapping layer 5 are ipsilaterally positioned about the primary wrapping layer 2, as depicted in FIG. 7. The ipsilateral positioning of the adhesive strip 12 and the secondary wrapping layer 5 ensures that the adhesive strip 12 is oriented towards the secondary wrapping layer 5 when the flap portion 4 is folded over the secondary wrapping layer 5 about the non-affixed edge 6.

In reference to FIG. 9, the adhesive strip 12 comprises an adhesive layer 13 and a protective layer 14. The adhesive layer 13 is adjacently connected to the flap portion 4, while the protective layer 14 is removably attached to the adhesive layer 13 opposite the flap portion 4. The adhesive layer 13 may be a double-sided adhesive, wherein the side of the adhesive layer 13 in contact with the flap portion 4 is permanently bonded to the primary wrapping layer 2. The protective layer 14 is a thin film that may be peeled off of the adhesive layer 13, such that the adhesive layer 13 may be adhered to the secondary wrapping layer 5. The protective layer 14 prevents the adhesive layer 13 from prematurely adhering to the secondary wrapping layer 5 or the tampon 17, while the wrapper body 1 is in the first configuration.

In reference to FIG. 1-2, the wrapper body 1 may further comprise a first tab 10 and a second tab 11 that assist the user in opening the primary wrapping layer 2 when the wrapper body 1 is in the first configuration. The first tab 10 and the second tab 11 are positioned opposite each other across the primary wrapping body, such that the first tab 10 and the second tab 11 are positioned adjacent to each other when the primary wrapping layer 2 is folded in the first configuration. The first tab 10 and the second tab 11 provide additional gripping surfaces, wherein the user can grasp the first tab 10 and the second tab 11 in separate hands and apply force in opposite directions in order to separate the first half 8 of the sealable perimeter 7 from the second half 9. The first tab 10 and the second tab 11 can be integrated with and/or connected to any of the primary wrapping layer 2, the secondary wrapping layer 5, or the sealable perimeter 7.

Preferably, the first tab 10 is positioned along the flap portion 4, opposite the pocket portion 3, while the second tab 11 is positioned along the pocket portion 3, opposite the flap portion 4, as depicted in FIG. 2. In this way, the first tab 10 and the second tab 11 are positioned parallel to the non-affixed edge 6, when the primary wrapping layer 2 is unfolded. The primary wrapping layer 2 being folded along an axis parallel to the non-affixed edge 6 prevents the adhesive strip 12 from being folded while the wrapper body 1 is in the first configuration. Thus, the integrity of the adhesive strip 12 is maintained, and the protective layer 14 is not compromised, such that the protective layer 14 remains attached to the adhesive layer 13 until removed by the user. In other embodiments, the first tab 10 and the second tab 11 may be positioned perpendicular to the non-affixed edge 6.

Furthermore, it is preferable that the first tab 10 and the second tab 11 are longitudinally offset from each other, as depicted in FIG. 7. The offset nature of the first tab 10 and the second tab 11 allows the user to readily identify the first tab 10 from the second tab 11 and determine the direction required to pull each tab in order to open the primary wrapping layer 2. Additionally, the offset nature of the first tab 10 and the second tab 11 makes it easier for the user to grasp each tab individually, as the first tab 10 and the second tab 11 cannot be pressed flush with each other, which would require the user to first separate the first tab 10 from the second tab 11 before opening the primary wrapping layer 2. However, in other embodiments, the first tab 10 and the second tab 11 may not be offset from each other.

Although the invention has been explained in relation to its preferred embodiment, it is to be understood that many other possible modifications and variations can be made without departing from the spirit and scope of the invention as hereinafter claimed.

What is claimed is:

1. A tampon disposal packaging comprises:
a wrapper body comprising a primary wrapping layer, a secondary wrapping layer, and a sealable perimeter;
the primary wrapping layer comprising a pocket portion and a flap portion;
the secondary wrapping layer comprising a non-affixed edge;
an adhesive strip;
the secondary wrapping layer being positioned adjacent to the pocket portion;
the sealable perimeter being perimetrically positioned about the primary wrapping layer;
the secondary wrapping layer being adjacently connected to the primary wrapping layer about the sealable perimeter to form a pocket;
the non-affixed edge delineating an opening to the pocket;
the non-affixed edge being positioned adjacent to the flap portion;
the adhesive strip being adjacently connected to the flap portion; and
the adhesive strip and the secondary wrapping layer being ipsilaterally positioned about the primary wrapping layer.

2. The tampon disposal packaging as claimed in claim 1 comprises:
the sealable perimeter comprising a first half and a second half;
the first half being folded over the second half to enclose the secondary wrapping layer and the adhesive strip within the primary wrapping layer; and
the first half being removably attached to the second half.

3. The tampon disposal packaging as claimed in claim 2 comprises:
the first half being tearably attached to the second half.

4. The tampon disposal packaging as claimed in claim 2 comprises:
the first half being adhered to the second half.

5. The tampon disposal packaging as claimed in claim 2 comprises:
the first half and the second half being heat sealed.

6. The tampon disposal packaging as claimed in claim 2 comprises:
a tampon being enclosed by the primary wrapping layer.

7. The tampon disposal packaging as claimed in claim 1 comprises:
the adhesive strip comprising an adhesive layer and a protective layer;
the adhesive layer being adjacently connected to the flap portion; and
the protective layer being removably attached to the adhesive layer opposite the flap portion.

8. The tampon disposal packaging as claimed in claim 1 comprises:
the flap portion being folded over the secondary wrapping layer; and
the adhesive strip being adhered to the secondary wrapping layer.

9. The tampon disposal packaging as claimed in claim 1 comprises:
the wrapper body further comprising a first tab and a second tab; and
the first tab and the second tab being positioned opposite each other across the primary wrapping body.

10. The tampon disposal packaging as claimed in claim 9 comprises:
the first tab being longitudinally offset from the second tab.

11. The tampon disposal packaging as claimed in claim 9 comprises:
the first tab being positioned along the flap portion, opposite the pocket portion; and
the second tab being positioned along the pocket portion, opposite the flap portion.

12. A tampon disposal packaging comprises:
a wrapper body comprising a primary wrapping layer, a secondary wrapping layer, and a sealable perimeter;
the primary wrapping layer comprising a pocket portion and a flap portion;
the secondary wrapping layer comprising a non-affixed edge;
an adhesive strip;
the secondary wrapping layer being positioned adjacent to the pocket portion;
the sealable perimeter being perimetrically positioned about the primary wrapping layer;
the secondary wrapping layer being adjacently connected to the primary wrapping layer about the sealable perimeter to form a pocket;
the non-affixed edge delineating an opening to the pocket;
the non-affixed edge being positioned adjacent to the flap portion;
the adhesive strip being adjacently connected to the flap portion;
the adhesive strip and the secondary wrapping layer being ipsilaterally positioned about the primary wrapping layer;
the wrapper body further comprising a first tab and a second tab;
the first tab and the second tab being positioned opposite each other across the primary wrapping body;
the adhesive strip comprising an adhesive layer and a protective layer;
the adhesive layer being adjacently connected to the flap portion; and
the protective layer being removably attached to the adhesive layer opposite the flap portion.

13. The tampon disposal packaging as claimed in claim 12 comprises:
the sealable perimeter comprising a first half and a second half;
the first half being folded over the second half to enclose the secondary wrapping layer and the adhesive strip within the primary wrapping layer; and
the first half being removably attached to the second half.

14. The tampon disposal packaging as claimed in claim 13 comprises:
the first half being tearably attached to the second half.

15. The tampon disposal packaging as claimed in claim 13 comprises:
the first half being adhered to the second half.

16. The tampon disposal packaging as claimed in claim 13 comprises:
the first half and the second half being heat sealed.

17. The tampon disposal packaging as claimed in claim 13 comprises:
a tampon being enclosed by the primary wrapping layer.

18. The tampon disposal packaging as claimed in claim 12 comprises:
the flap portion being folded over the secondary wrapping layer; and
the adhesive strip being adhered to the secondary wrapping layer.

19. The tampon disposal packaging as claimed in claim 12 comprises:
the first tab being longitudinally offset from the second tab.

20. The tampon disposal packaging as claimed in claim 12 comprises:
the first tab being positioned along the flap portion, opposite the pocket portion; and
the second tab being positioned along the pocket portion, opposite the flap portion.
